(19) United States
(12) Patent Application Publication
(10) Pub. No.: US 2005/0131504 A1
(43) Pub. Date: Jun. 16, 2005

(54) Beauty mask

(76) Inventor: Myeong-Ha Kim, Gimpo-si (KR)

Correspondence Address: Diane Dunn McKay, Esq.;Mathews, Collins, Shepherd & Gould, P.A., Suite 306, 100 Thanet Circle, Priceton, NJ, 08540, US

(21) Appl. No.: 10/731,990

(22) Filed: Dec. 10, 2003

(51) Int. Cl. A61F 7/00

(52) U.S. Cl. 607/104

(57) ABSTRACT

The present invention is directed to a beauty mask having a circulator using a thermoelectric element, more specifically the beauty mask in which a urethane pack filled with silicon oil is attached on inner surface of a face mask, a tube allowing the silicon oil cooled or heated by the thermoelectric element to flow in or out the pack is connected to the urethane pack, and the urethane pack is cooled or heated according to a temperature of the inflow silicon oil and provides cold and hot packing effect to the face, so that a metabolism of a face skin is facilitated, the resilience of the skin is improved, and wrinkle can be eliminated. By using of the beauty mask according to the present invention, an aging phenomenon of the skin can be suppressed, waste material stuck in a hypodermis of a facial skin can be discharged therefrom, and a blood circulation of the facial skin is smoothly achieved due to cold and hot packing effect

### BACKGROUND OF THE INVENTION

1. Field of the Invention

The present invention is related to a beauty mask having a circulator using a thermoelectric element, more specifically the beauty mask in which a urethane pack filled with silicon oil is attached on inner surface of a face mask, a tube allowing the silicon oil cooled or heated by the thermoelectric element to flow in or out the pack is connected to the urethane pack, and the urethane pack is cooled or heated according to a temperature of the inflow silicon oil and provides cold and hot packing effect to the face, so that a metabolism of a face skin is facilitated, the resilience of the skin is improved, and wrinkle can be eliminated.

2. Background of the Related Art

From one's late twenties, aging phenomena is initiated and wrinkles are developed in his or her skin. Especially, since a hypodermis of a facial skin is relatively thin as compared with that of other parts of the human body, the wrinkles are formed more rapidly, and since the hypodermis is the most exposed part in the human body, it sensitively reacts according to a change of circumstance. Accordingly, for a skin aging prevention and constant skin resilience, required is a correct makeup and proper massage suitable to one's skin condition together with a skin cleaning. In addition, if one's face is primarily washed with warm water before the makeup and massage, the skin resilience is decreased with pores of the skin expanded, and at this time, the skin resilience is maintained so as to prevent the aging by decreasing a temperature of the skin with cold water and the like.

In the prior art, in order to suppress such aging phenomena or eliminate waste material remaining in the hypodermis of the facial skin, one wears a mask after coating functional material such as massage cream, nutrition cream and the like on the face, or wears a massage mask as disclosed in Korean Patent 291011(Mar. 7, 2001) on the face.

Recently, Korean Patent Application No. 10-1998-0038229 has been disclosed a beauty mask coated with material radiating infrared rays or a beauty mask having magnets therein so as to help a blood circulation by a magnetic filed.

Most of the beauty masks perform any one of cold packing (i.e. a cold fomentation or a cold compress) or a hot packing (i.e. a hot fomentation or a hot compress). For such cold or hot packing, a pack filled with a predetermined material becomes cold or hot by putting it in a refrigerator or a microwave oven for approximately 2-3 hours or 2-3 minutes, and then this pack is worn on the one's face for the massage. However, a packing method as above can not have the effect of both cold and hot massages such as alternate cold bath and hot bath.

### SUMMARY OF THE INVENTION

The present invention is contemplated to solve the aforementioned problem, and it is an object of the present invention to provide a beauty mask for repeatedly cooling and heating a face and thus providing both of cold packing and hot packing in order to facilitate a metabolism of a skin and increase resilience of the skin.

To accomplish the above object, the present invention provides a beauty mask comprising: a face mask formed to be a shape corresponding to a user's face using flexible plastic material; a pack have a shape corresponding to that of the face mask and is attached on an inner surface of the face mask, the pack is formed with a path in which a predetermined circulation medium circulates; a circulator controlling cooling and heating of the circulation medium by using a thermoelectric element; and a tube having an one end connected to the pack and the other end connected to the circulator, the tube allowing the circulation medium to flow in the pack, circulate in the path, and flow out of the pack.

Preferably, the tube includes an inflow conduit and an outflow conduit accommodated in a single protection cover, and silicon oil is used as the circulation medium.

The circulator comprises: a pump disposed between an inflow conduit and an outflow conduit in the tube and generating circulation force for the circulation medium by pumping it; the thermoelectric element disposed to conduits through which the circulation medium flowing out of the pump passes and controlling a temperature of the circulation medium by heating or cooling the medium according to current directions provided thereto; a fan for discharging hot or cool air accumulated in a housing of the circulator by the cooling or heating of the thermoelectric element; a fan heater disposed near the thermoelectric element, the fan heater generating heat using the hot wire therein and blowing heated air to the thermoelectric element so as to preventing dew on the thermoelectric element in an initial operation of the thermoelectric element; and a controller providing the current changing the direction thereof according preset cold or hot operation, controlling the circulation medium to have the circulation force by transmitting the signal for controlling an operation of the pump to the pump, and providing power for operating the fan and fan heater.

An inner surface of the pack is coated with antibiotic material to have antibiotic and auxiliary cap function and is coated with bio-ceramic material to have sterilization, secondary dissolving toxin and anti-mold function.

The circulation medium is configured to be cooled or heated in a range of 20° C.-45° C. in order to perform cold or hot packing repeatedly so that a metabolism of a face skin is facilitated and the resilience of the skin is improved.

### BRIEF DESCRIPTION OF THE DRAWINGS

The features and advantages of the present invention will be more described specifically in the following description of preferred embodiments of the invention with reference to the accompanying drawings wherein:

FIG. 1 is an exploded perspective view showing a preferred embodiment according to the present invention;

FIG. 2 is a block diagram showing a configuration of a circulator according to the present invention;

FIG. 3 is a sectional view showing a section of a beauty mask according to the present invention;

FIG. 4 is a sectional view showing a path in a urethane pack according to the present invention; and

FIG. 5 is a perspective view showing an example of usage of the beauty mask according to the present invention.

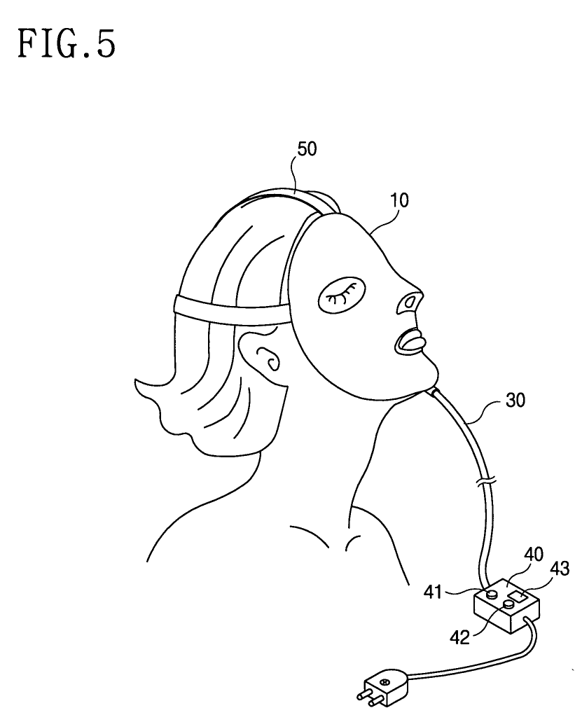

### DETAILED DESCRIPTION OF THE PREFERRED EMBODIMENT

Reference will now be made in detail to a embodiment of the present invention, examples of which are illustrated in the accompanying drawings. In explaining the present invention, the same names and reference numerals will be given to the same components, and explanations in the same will be omitted.

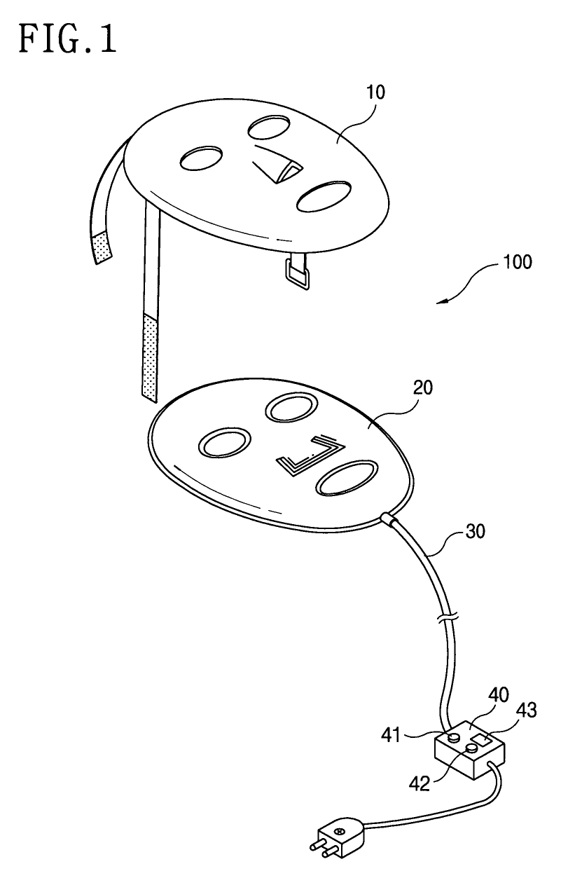

First, referring to FIG. 1 showing a perspective view, a preferred embodiment of the present invention will be explained.

A face mask 10 of FIG. 1 is formed to be a shape corresponding to a user's face using flexible plastic material, and formed to expose eyes, nose, and mouse when the user wear it on the face, in order to secure the user's sight and breathing.

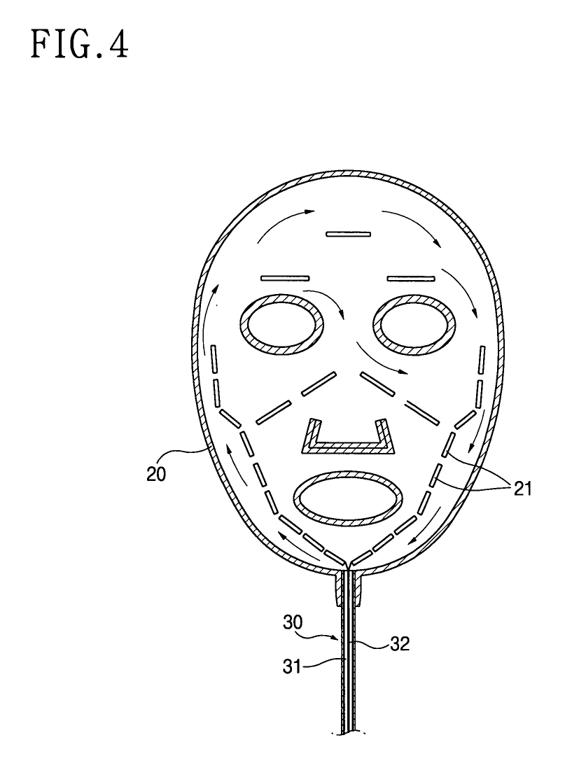

A pack 20 made of urethane material of which eyes, nose, and mouse portions are perforated so as to have a shape corresponding to that of the face mask 10, is fixed on an inner surface of the face mask 10 as a laminate, and as shown in FIG. 4, a path is formed in an inner space of the pack 20.

A tube 30 for an inflow and an outflow of a circulation medium is connected to a portion corresponding to the jaws in the pack 20 to be inserted into the pack 20, and a connection portion of the tube 30 and the pack 20 is sealed with silicon material in order to prevent the circulation medium from leaking.

A surface of the pack 20 come in contact with the face is coated with antibiotics such as bio-ceramic material.

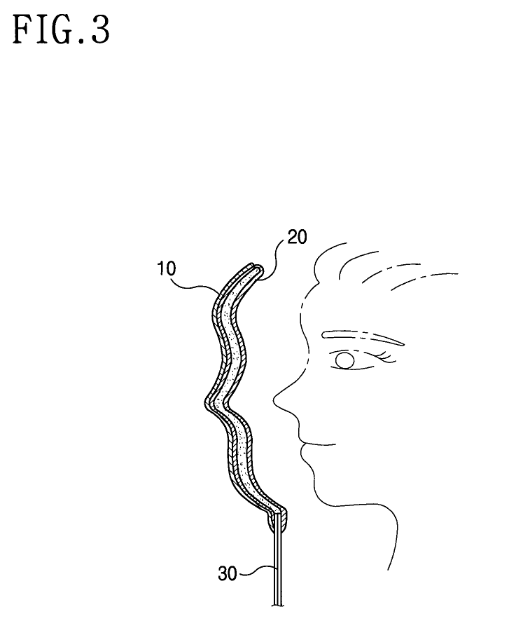

A sectional view in FIG. 3 shows cross sections of the face mask 10 and the pack 10 attached thereto well, and it is noted that a space is formed for circulating silicon oil flowing into the pack 20 through the tube 30.

In addition, according to FIG. 4 showing a path in the urethane pack 20, shown are projections 21 for forming the path such that the silicon oil input to the pack 20 through the tube 30 circulates uniformly along the path in the pack 20, and arrows shown therein are to a flowing direction of the silicon oil.

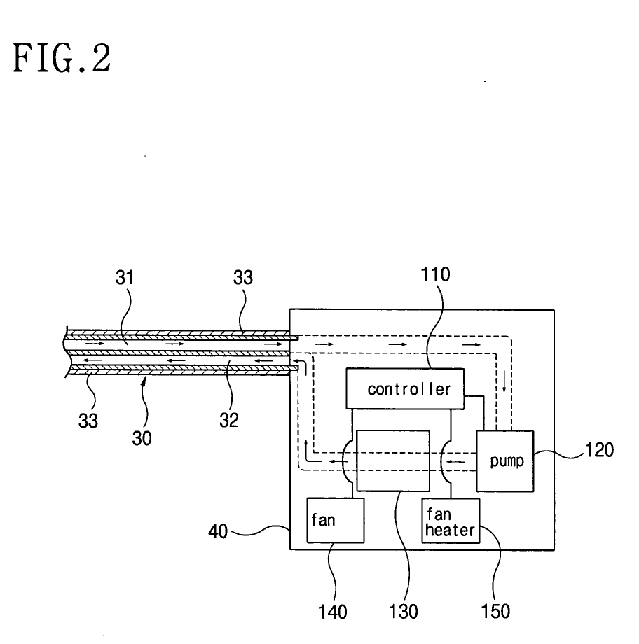

The tube 30 is enclosed by a protection cover 33 to include an inflow conduit 31 and an outflow conduit 32 together. A one end of the tube 30 is inserted into the pack 20 as mentioned above, and the other end thereof is connected to a circulator to be described below so that cooling and heating adjustment is available by a circulation of the circulation medium as shown in FIG. 2.

The present invention preferably uses the silicon oil as the circulation medium, and a range of temperature of the silicon oil controlled by a controller 110 in the circulator 40 is set as 20° C.-45° C.

In addition, as shown in FIG. 1, the circulator 40 includes a power switch 41, a function selection switch 42, and a display 43 installed on an outer surface of the housing thereof. The power switch 41 is configured to provide the power to the circulator 40, the function selection switch 42 is configured to select various preset functions such as a cold packing, a hot packing or an automatic cold and hot packing adjustment, and the display 43 is comprised of a liquid crystal display or a light emitting diode to show the temperature/operation time of the silicon oil and the pack 20 controlled by the circulator 40, the present time, the present operation status and the like.

FIG. 2 is a block diagram showing a configuration of the circulator 40 according to the present invention.

The circulator 40, as shown in FIG. 2 comprises the controller 110, a pump 120, a thermoelectric element 130, a fan 140, and a fan heater 150 accommodated in the housing connected with the other end of the tube 30. Such circulator 40 will be explained in detail as follows.

On a flowing path of the silicon oil as the circulation medium which is input into the circulator 40 through the inflow conduit 31 of the tube 30, the pump 120 configured to be controlled by the controller is provided.

The pump 120 is a water circulation type pump, and is configured to pump the silicon oil input through the inflow conduit 31 to the outflow conduit 32.

The thermoelectric element 130 is an element which uses a thermoelectric effect (so called a Peltier effect) converting an electric energy into a thermal energy by movement of electric charge carriers using an energy level difference between metal and semiconductor. When a polarity of provided voltage is changed, the thermoelectric element 130 generates the phenomenon producing heat or absorbing heat so that it is configured to achieve cooling or heating without a refrigerant.

Such thermoelectric element 130 is dispose to the outflow conduit 32 extending from the pump 120 so as to the silicon oil in the outflow conduit 32 using the provided power, and is sealed with an aluminum-die casting cover to protect it from dewing which can occur according to a sudden change in temperature.

The fan 140 for discharging hot or cool air accumulated in the housing by the cooling or heating of the thermoelectric element 130, is connected to the controller 110 so that it is configured to discharge the air in the housing.

In addition, the fan heater 150 is an additional element to more safely protect the thermoelectric element 130 which is primarily protected from the dewing by the aluminum-die casting cover. The fan heater 150 is disposed near the thermoelectric element 130 and is configured to blow hot air for approximately 2-3 seconds in order to eliminate dew drops on the thermoelectric element 130 when the circulator 40 operates in the first place. Therefore, in an initial operation, the thermoelectric element 130 is prevented from the primary dewing.

The controller 140 is comprised of a microcomputer, and receives a signal for cold packing, hot packing or adjusting the automatic cold and hot packing from the function selection switch 42 and then transmits a signal for cooling, heating or repeating the cooling and heating to the thermoelectric element 130 according to the receiving signal. Also, the controller 140 instantly operates the fan heater 150 to eliminate the dew on the thermoelectric element 130 before transmitting the signal to the thermoelectric element 130.

The controller 110 is configured to operate the pump 120 to circulate the silicon oil through the outflow conduit 32, the tube 30, the pack 20, the path in the pack, the inflow conduit 31, and the pump 120 again, when the silicon oil in the outflow conduit 32 is cooled, heated or repeatedly cooled and heated by the thermoelectric element 130.

An operation of the beauty mask 100 having the circulator as mentioned above will be described in detail as follows.

After the user wears the beauty mask 100 on the face, the user turns on the mask 100 by operating the power switch 41 of the circulator 40, and then selects a desired function using the function selection switch 42.

The cold packing, hot packing, and the cold and hot packing are available in the circulator 40, and the case the user selects the cold and hot packing will be explained, omitting the cold or hot packing for performing the cooling or the heating merely.

When the user selects the cold and hot packing function, the controller 110 eliminates the dew on the thermoelectric element 130 by operating the fan heater 150 for approximately 2-3 seconds, and then provides the silicon oil used as the circulation medium with circulation force by providing the power to the pump 120.

The silicon oil provided with the circulation force initiated to circulate through the outflow conduit 32, the circulated silicon oil is cooled or heated passing through the thermoelectric element 130 and is inputted to the tube 30, and then the inputted silicon oil flows into the urethane pack 20 through the outflow conduit 32.

The silicon oil flowed into the pack 20 smoothly circulates in the inner space of the pack 20, guided by the projections 21 formed in the pack 20, and is inputted into the circulator 40 after flowing out of the pack through the inflow conduit 32.

Through these procedures, the temperature of the silicon oil in the urethane pack 20 is set to the temperature of the cold and hot packing desired by the user. For example, if the operation of the mask 100 is set in order of the cold packing and then hot packing, the silicon oil is cooled by 20° C. and is kept in this temperature for several or tens of minutes, and then is heated by 45° C. and is kept in such temperature for several or tens of the minutes. These procedures are repeatedly performed.

When such cold and hot packing are repeatedly performed, the cold and hot packing effects are repeatedly provided to the face of the user, so that a metabolism of a face skin is facilitated, the resilience of the skin is improved, and wrinkle can be eliminated.

The advantages of the beauty mask according to the present invention are summarized as follows.

As the beauty mask according to the present invention uses the circulator with the thermoelectric element performing the cooling or the heating according the direction of the provided power, the user can conveniently use this beauty mask without directly cooling or heating it by putting it into a refrigerator or a microwave oven.

An aging phenomenon of the skin can be suppressed and waste material stuck in a hypodermis of a facial skin can be discharged therefrom by a smooth circulation caused by the cold and hot packing.

In addition, as a blood circulation of the facial skin is smoothly achieved due to the convenient cold and hot packing effect, the resilience of the skin is restored and thus the wrinkles are eliminated.

Although a number of embodiment have described in the above specification, it should be apparent that the present invention could be embodied in many other specific mode included within the sprit and scope of the present invention. Thus, the present embodiments should be considered as illustrative, and the present invention could be modified within the scope of claims and the equivalent thereof.

1. A beauty mask comprising:
a face mask formed to be a shape corresponding to a user's face using flexible plastic material;
a pack have a shape corresponding to that of the face mask and is attached on an inner surface of the face mask, the pack is formed with a path in which a predetermined circulation medium circulates;
a circulator controlling cooling and heating of the circulation medium by using a thermoelectric element; and
a tube having an one end connected to the pack and the other end connected to the circulator, the tube allowing the circulation medium to flow in the pack, circulate in the path, and flow out of the pack.

2. The beauty mask according to claim 1, wherein the circulation medium is silicon oil.

3. The beauty mask according to claim 1, wherein the circulator comprises:
a pump disposed between an inflow conduit and an outflow conduit in the tube and generating circulation force for the circulation medium by pumping it;
the thermoelectric element disposed to conduits through which the circulation medium flowing out of the pump passes and controlling a temperature of the circulation medium by heating or cooling the medium according to current directions provided thereto;
a fan for discharging hot or cool air accumulated in a housing of the circulator by the cooling or heating of the thermoelectric element;
a fan heater disposed near the thermoelectric element, the fan heater generating heat using the hot wire therein and blowing heated air to the thermoelectric element so as to preventing dew on the thermoelectric element in an initial operation of the thermoelectric element; and
a controller providing the current changing the direction thereof according preset cold or hot operation, controlling the circulation medium to have the circulation force by transmitting the signal for controlling an operation of the pump to the pump, and providing power for operating the fan and fan heater.

4. The beauty mask according to claim 3, wherein the circulation medium is cooled or heated in a range of 20° C.-45° C.
